(19) United States
(12) Patent Application Publication
(10) Pub. No.: US 2016/0136606 A1
(43) Pub. Date: May 19, 2016

(54) CAPROLACTAM-BASED FATTY AMIDE AS GELLING ADDITIVE

(71) Applicant: ARKEMA FRANCE, Colombes (FR)

(72) Inventor: Michael Y. BERNARD, Enghien Les Baines (FR)

(21) Appl. No.: 14/899,159

(22) PCT Filed: Jun. 16, 2014
(86) PCT No.: PCT/FR2014/051472

§ 371 Date: Dec. 17, 2015

(30) Foreign Application Priority Data
Jun. 28, 2013 (FR) 1356319

(51) Int. Cl. B01J 13/00; C11D 3/32; A61Q 19/00; C09J 11/06; D06M 13/402; A61K 8/42; C09D 7/00

(52) U.S. Cl. CPC B01J 13/0065; A61K 8/42; C11D 3/32; C09D 7/002; C09J 11/06; D06M 13/402; A61Q 19/00; A61K 2800/10; A61K 2800/48; A61K 2800/591

(57) ABSTRACT

The present invention relates to a caprolactam-based fatty amide additive having an amine or salified amine ending useful as a hydrogelator for aqueous compositions.

The present invention relates to a fatty amide additive having an amine or salified amine ending and used as gelator for organic or aqueous media, in particular as hydrogelator for aqueous media, such as coatings or adhesives or treatment of fibres or textiles or for detergents, stripping agents, depolluting agents or flocculating agents.

Various systems which can gel in water or in an organic solvent with respectively hydrogelator and organogelator additives are already known and used in various applications.

FR 2 976 948 describes a ternary combination of an acid of boron, such as boric acid, with an N-alkylaldonamide, in particular N-dodecyl-D-glucoanamide, and a monovalent salt, such as NaCl, for the achievement of gels in a (saline) aqueous medium for various applications and in particular in detergency.

The known hydrogelators include those based on lysine derivatives, such as described by M. Suzuki et al. in Chem, Soc. Rev., 2009, 38, 967-975, where derivatives used as organogelators are also described. A description is given, among these derivatives, of diurea-esters, amide-esters or amide-urea-esters, starting from lysine.

A review of hydrogelators has been carried out by L. A. Estroff et al. in Chemical Reviews, 2004, 104, 3, 1201-1217, with an inventory of the characterization methods and structures which are known. Conventional amphiphilic derivatives having a hydrophilic head and one or two hydrophobic chains, bolaamphiphiles having two hydrophilic heads bonded via a hydrophobic chain, surfactants having a double ionic head separated by a rigid spacer with two flexible end chains, systems derived from sugars, are singled out, inter alia. It is recognized that there is no rule generally applicable which makes it possible to find a satisfactory compromise between hydrophilicity and hydrophobicity of a molecule and consequently there is no general rule either between the ability to form a gel in an aqueous medium and the tendency to prevent the precipitation of the fibres.

The disadvantage of these hydrogelators or organogelators is the fact that they are based on amino acids which can undergo side reactions and in particular uncontrolled chain elongations according to the conditions for the preparation of the said gelators and can thus affect their fine structure and consequently their performance of gelator. Furthermore, none of the documents cited describes or teaches how to obtain amide additives modified by caprolactam and with an improved performance, a subject-matter of the present invention, in order to overcome the disadvantages of the state of the art.

Specifically, the present invention attempts to develop novel amide additives modified by a caprolactam structure (equivalent to a C₆ amino acid) without having recourse to amino acids or to acids and to amines which react by polycondensation and in several stages requiring acyl chlorides.

These novel amides must make possible the use of a preparation process which is simple and practical to carry out, with the controlled opening of the ring of the caprolactam, in order to prevent side reactions which are difficult to avoid with an equivalent amino acid or by acid/amine condensation and without needing stages of separation and/or purification of the final product. The said novel amides must exhibit a satisfactory gelator rheological performance in an organic or aqueous medium and in particular in an aqueous medium, without affecting the specific performances of the binders, in particular aqueous, which may be used in association with them.

The first subject-matter of the invention is a gelator additive based on fatty amide which is the product of the addition reaction (without elimination of by-products) of a fatty amine with caprolactam in a controlled specific ratio with a specific composition.

Thus, the first subject-matter of the invention is a gelator additive based on fatty amide which is the product of the addition reaction of a fatty amine R₁NH₂ with caprolactam with an end amine functional group, either in the amine form or in the salified ammonium salt form, by neutralization of the said amine functional group by a neutralizing agent, and which (the said additive) consists of or comprises the mixture of 3 different amide compounds resulting from the said reaction (condensation) and which are characterized by the number n of incorporated caprolactam units, which is respectively 1, 2 and 3, with a mean number n of units (mean per molecule) ranging from 0.8 to 3, preferably from 0.9 to 2.75 and more preferably from 0.9 to 2.5. More particularly, the said 3 amide compounds can be defined according to the formula (I) and respectively correspond to n=1, n=2 and n=3

R₁—NH—[—C(═O)(CH₂)₅NH]ₙ₋₁—C(═O)(CH₂)₅—Y (I)

with Y being —NH₂ or —NH₃⁺+X⁻, if the amine is in the salified form and with X⁻ being an organic or inorganic counteranion related to the acid neutralizing agent used, X—H.

Preferably, the said fatty amine R₁NH₂ comprises a number of carbon atoms ranging from 10 to 24, which means that R₁ is a C₁₀ to C₂₄ alkyl, and R₁ is preferably linear,

Mention may be made, as suitable examples of R₁NH₂, of fatty monoamines, such as decylamine, undecylamine, dodecylamine (or laurylamine), tridecylamine, tetradecylamine, pentadecylamine, hexadecylamine, heptadecylamine, octadecylamine (or stearylamine), eicosanamine, doeicosanamine or tetraeicosanamine, or their isomers and their mixtures. More particularly, the said amine functional group can be in the salified form, in particular for use as hydrogelator, and the said neutralizing agent used is selected from organic or inorganic acids.

Mention may be made, as suitable examples of inorganic acid, inter alia, of hydrochloric acid, sulphuric acid, phosphoric acid, boric acid or nitric acid.

Mention may be made, as suitable examples of organic acids, of carboxylic acids, sulphonic acids, phosphonic acids and phosphinic acids.

The said additive of the invention can be used in particular in the gel form in water or in the gel form in an organic solvent, preferably at a content by weight of less than 5% and more preferably at a content not exceeding 1% by weight, this percentage being defined with respect to the weight of the water+additive, and more preferably still the said additive is used as hydrogelator in the gel form in water or, in other words, in the hydrogel form.

The said additive can be in the form of a micronized powder, preferably having a volume-average size of less than 50 μm, preferably of less than 25 μm. This particle size distribution can be determined directly on the dry powder by laser diffraction. This technique is based on the principle that particles passing through a laser beam diffract the light along a different angle as a function of their size: the particles having small sizes diffract at large angles, whereas the particles having larger sizes diffract at small angles.

The said organic solvent is preferably a polar organic solvent or an organic solvent which is a homogeneous mixture without phase separation and which comprises a polar organic solvent, such as a C₁ to C₄ alcohol, dimethyl sulphoxide (DMSO) or dimethylformamide (DMF), N-methylpyrrolidone (NMP), N-ethylpyrrolidone (NEP) or a polar organic plasticizer.

The second subject-matter of the invention is a process for the preparation of the gelator additive according to the invention, which process comprises a stage of an addition reaction between a fatty amine R₁NH₂ and caprolactam with a molar ratio of the said caprolactam with respect to the said fatty amine ranging from 0.8 to 3, preferably from 0.9 to 2.75 and more preferably from 0.9 to 2.5, the said process not comprising any separation or purification stage. This reaction is a bulk addition reaction in the molten state, which can take place at a temperature ranging from 200° C. to 300° C. and under an inert atmosphere. After cooling, the product obtained is micronized by mechanical grinding or an air jet. A sieving can make it possible to obtain a fine and controlled particle size distribution with a volume-average size of less than 50 μm, preferably less than 25 μm, measured by laser diffraction, such as, for example, on the Mastersizer® S from Malvern. A catalyst can be used for this reaction, such as, for example, a Lewis acid or a Lewis base.

Another subject-matter of the invention is an aqueous composition or a composition in an organic solvent medium, preferably an aqueous composition, which comprises at least one additive as defined above or obtained by the process as defined above according to the invention, the said additive being used as gelator and in particular for an aqueous composition as hydrogelator and more particularly as rheology additive. Preferably, the said composition is aqueous and has an acidic pH preferably of less than 5, more preferably of less than 4.

This pH is also valid for the additive present in the gel form in water (hydrogel).

This pH can be adjusted by addition of the said organic or inorganic acid in excess.

According to a more specific option, the said composition is an aqueous organic binder composition and in particular a coating composition, more particularly as regards a coating composition from varnishes, paints or inks or an adhesive or cosmetic composition or an aqueous composition for the treatment of fibres or textiles and the said hydrogelator additive is a rheology additive. In this composition option, the said aqueous composition is an organic binder composition and thus comprises, in addition to the said additive, at least one organic binder related to the targeted application. According to another aqueous composition option, this composition may not comprise an organic binder and may be a composition of the said additive in water in the hydrogel form or it can be a surfactant, in particular detergent, or stripping agent or depolluting agent or flocculating agent composition.

In the case where the said additive is used as organogelator in an organic solvent medium (or an organic medium), preferably a polar medium, the said composition in an organic medium which results therefrom can be a composition of the said additive in the said solvent in the organogel form or it can be a coating composition, in particular from paints, varnishes, inks or gel coats, or an adhesive composition or a mastic or leaktightness agent or stripping agent composition or a moulding composition.

The aqueous compositions comprising the said additive as hydrogelator are more particularly preferred.

The invention also relates to the use of a fatty amide additive as defined above according to the invention as gelator additive for compositions in an organic solvent medium, as organogelator, or for aqueous compositions, as hydrogelator. More particularly, the said additive is used as hydrogelator in water, in order to obtain a hydrogel, or in an aqueous detergent, depolluting agent, stripping agent or flocculating agent composition or in an aqueous coating or adhesive or cosmetic composition or an aqueous composition for the treatment of fibres or textiles. The aqueous coating composition is particularly preferred where the said hydrogelator additive is used as rheology additive.

The said additive can also be used, in particular in the nonsalified form, as organogelator in an organic solvent, preferably a polar solvent, in order to obtain an organogel, or in a composition in an organic solvent medium, in particular in a coating composition from paints, varnishes, inks or gel coats, or in an adhesive composition or in a cosmetic composition or a moulding composition or a mastic or leaktightness agent or stripping agent composition.

The gel strength or the gelling intensity depends on and can be adjusted according to the application targeted and the medium used.

The invention also covers a gel from organogel or hydrogel, in particular hydrogel, which is the product resulting from the use of the said additive. It is an organogel in an organic solvent or medium or a hydrogel in water or in an aqueous medium.

Finally, the final product resulting from the use in an aqueous medium, as hydrogelator and more particularly as rheology additive, of at least one additive as defined above or obtained by a process as defined above according to the invention, and in particular the said final product being selected from coating or adhesive or cosmetic or detergent or stripping agent or depolluting agent or treated fibre or treated textile, also come within the invention.

The following examples are presented by way of illustration of the invention and of its performance and, for this reason, do not in any way limit its coverage.

### EXPERIMENTAL PART

### I—Starting Materials Used

TABLE 1
Starting materials used

| Product | Function | Commercial reference | Supplier |
|---|---|---|---|
| Octadecylamine | Reactant | Octadecylamine 97% | Aldrich |
| Caprolactam | Reactant | ε-Caprolactam | Aldrich |
| Zinc carboxylate | Catalyst | Borchikat ® 22 | OMG Borchers |
| | | | SAS |
| Hydrochloric acid | Acid | Hydrochloric acid | Aldrich |
| | | ACS reagent, 37% | |

### II—Examples of the Preparation of Caprolactam-Based Hydrogelator Amides

### Example 1

### Preparation of the Amide A1 by Reaction of 1 mol of Octadecylamine with 1 mol of Caprolactam

113.16 g of caprolactam (1 mol), 274.84 g of octadecylamine (1 mol) and 1.95 g of Borchikat® 22 are introduced, under a stream of nitrogen, into a 1 litre reactor equipped with a thermometer, a condenser and a stirrer.

The mixture is heated to 250° C., still under a stream of nitrogen. The reaction is controlled by the viscosity. After 15 hours, the viscosity value becomes unvarying (and >0.23 P or >23 mPa·s, measured on a Brookfield® CAP1000 at 100° C.), the reaction mixture is cooled to 150° C. and then it is discharged into a silicone mould. Once cooled to ambient temperature, the product is micronized mechanically by grinding. A sieving is carried out in order to obtain a fine and controlled particle size distribution with a mean size obtained of 7 μm.

### Example 2

### Preparation of the Amide A2 by Reaction of 1 Mol of Octadecylamine with 2 Mol of Caprolactam

226.32 g of caprolactam (i.e., 2 mol), 274.84 g of octadecylamine (i.e., 1 mol) and 1.95 g of Borchikat® 22 are introduced, under a stream of nitrogen, into a 1 litre reactor equipped with a thermometer, a condenser and a stirrer.

The remainder of the procedure is carried out as described above for Example 1.

### III—Evaluation of the Gelling Performance

### 1. Formulation for the Evaluation of the Amide Additives as Hydrogelators

99 grams of demineralized water, 1 g of ground or micronized hydrogelator amide additive to be tested and then a few drops of hydrochloric acid with an excess corresponding to more than 4 times the amine equivalents of the hydrogelator amide to be tested are introduced into an Erlenmeyer flask equipped with a magnetic bar. The Erlenmeyer flask is subsequently closed. The mixture is then stirred for more than 5 hours at 85° C. in order to have complete dissolution of the hydrogelator and a milky but precipitate-free mixture. Finally, the mixture is introduced into a test tube and then left standing at 25° C. for 24 hours.

Three formulations were prepared under these preparation conditions with the amide A1 and the amide A2 as described respectively in Preparation Examples 1 and 2 and also octadecylamine as comparison reference. These 3 tests are summarized in Table 2 below.

TABLE 2
Formulations

| | Hydrogelator amide additive | % by weight of |
|---|---|---|
| | or reference comparative | additive in aqueous |
| Test ref. | additive | formulation |
| 1 | Octadecylamine (comparative) | 1% |
| 2 | Amide A1 (invention) | 1% |
| 3 | Amide A2 (invention) | 1% |

### 2. Evaluation of the Gel

The 3 tests on formulations were evaluated in two ways: first according to the appearance of the formulations prepared in the test tubes after 24 hours (see Table 3) and subsequently according to their viscosities at different shear rates on a Brookfield® viscometer (see Table 4).

TABLE 3
Appearance

| Test ref | Appearance |
|---|---|
| 1 | Suspension |
| 2 | Gel |
| 3 | Gel |

TABLE 4
Viscosity at different rates

| | Spindle speed (rpm) | Test 1 | Test 2 | Test 3 |
|---|---|---|---|---|
| Viscosity | 1 | 3200* | 6700 | 3300 |
| measured with | 5 | 900* | 1860 | 1440 |
| Brookfield at | 10 | 530* | 1080 | 870 |
| 25° C. (mPa · s) | 50 | 174* | 390 | 238 |
| | 100 | 116 | 250 | 126 |

*Comment: the results are not very repeatable (reproducible), the formulation not being homogeneous

In contrast to octadecylamine, the formulations with amides A1 or A2 according to the invention exist in the gel form characteristic of a hydrogelator.

With regard to the viscosity results, they show that the formulation comprising the amide A1 (Test 2) is thixotropic but also more viscous than water or the formulation comprising octadecylamine.

1. Gelator additive based on fatty amide which is a product of an addition reaction of a fatty amine R₁NH₂ with caprolactam with an end amine functional group in amine form or in salified ammonium salt form, by neutralization of said amine functional group by a neutralizing agent, wherein said additive comprises a mixture of 3 different amide compounds resulting from said reaction and which are characterized by the number n of incorporated caprolactam units, which is respectively L 2 and 3, with a mean number II of units (mean per molecule) ranging from 0.8 to 3.

2. Additive according to claim 1, wherein said 3 amide compounds are defined according to the following formula (I) and respectively correspond to n=1, n=2 and n=3
R₁—NH—[—C(═O)(CH₂)₅NH]ₙ-1-C(═O)(CH₂)₅—Y (I)
with Y being —NH₂ or —NH₃⁺X⁻, if the amine is in salified form and with X⁻ being an organic or inorganic counteranion related to the acid neutralizing agent used, X—H.

3. Additive according to claim 1 wherein said fatty amine comprises a number of carbon atoms from 10 to 24.

4. Additive according to claim 1 wherein said amine functional group is in salified form and said neutralizing agent is selected from the group consisting of organic and inorganic acids.

5. Additive according to claim 1 wherein said additive is a micronized powder having a volume-average size of less than 50 μm.

6. Additive according to claim 1 wherein it is in gel form in water or in gel form in organic solvent, at a content by weight of less than 5% by weight.

7. Process for the preparation of an additive according to claim 1 comprising a stage of an addition reaction between fatty amine R₁NH₂ and caprolactam with a molar ratio of said caprolactam with respect to the said fatty amine from 0.8 to 3 said process not comprising separation or purification stages.

8. An aqueous composition or a composition in an organic solvent medium comprising at least one additive according to claim 1.

9. Composition according to claim 8 which is an aqueous composition having a pH of less than 5.

10. Composition according to claim 8 which is an aqueous surfactant.

11. Aqueous composition according to claim 8 which is an aqueous organic binder composition, a coating, an adhesive a cosmetic composition, or an aqueous composition for the treatment of fibres or textiles.

12. (canceled)

13. Composition according to claim 8, which is a composition in an organic solvent in the organogel form, or is a coating composition, an adhesive composition, a mastic or leaktightness agent, stripping agent composition, or a moulding composition.

14. (canceled)

15. (canceled)

16. (canceled)

17. (canceled)

18. (canceled)
